(19) United States
(12) Patent Application Publication
(10) Pub. No.: US 2019/0216786 A1
(43) Pub. Date: Jul. 18, 2019

(54) USE OF 2-{4-[(4-{[1-CYCLOPROPYL-3-TETRAHYDRO-2H-PYRAN-4-yl)-1H-PYRAZOL-4yl]OXY} PYRIDINE-2-yl)AMINO]PYRIDIN-2-yl}PROPAN-2-OL IN THE TREATMENT OF CANCER

(71) Applicant: Eli Lilly and Company, Indianapolis, IN (US)

(72) Inventors: Karim Adnane BENHADJI, Green Village, NJ (US); Kyla Elizabeth DRISCOLL, Indianapolis, IN (US); Michael Mauritius Fabio LAHN, Oberursel (DE); Jason Scott SAWYER, Indianapolis, IN (US); Anja Jamandre STAUBER, Carmel, IN (US)

(21) Appl. No.: 16/333,836

(22) PCT Filed: Sep. 22, 2017
(86) PCT No.: PCT/US2017/052864

§ 371 Date: Mar. 15, 2019

Related U.S. Application Data
(60) Provisional application No. 62/402,118, filed on Sep. 30, 2016.

(51) Int. Cl. A61K 31/444; A61P 35/00

(52) U.S. Cl. CPC A61K 31/444; A61P 35/00

(57) ABSTRACT

The present invention relates to novel methods of using 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol or a pharmaceutically acceptable salt thereof to treat physiological conditions such as cancer.

The present invention relates to novel methods of using 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol or a pharmaceutically acceptable salt thereof, in particular 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate to treat physiological conditions such as cancer.

TGFβ signaling has been linked in the literature to several physiological conditions such as cancer. 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol and pharmaceutically acceptable salts thereof, in particular 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate are disclosed in WO2016/057278 as small molecule inhibitors of the TGF-β receptor I kinase.

There is a need for new methods of treating conditions such as cancer that may maximize efficacy while continuing to minimize patient toxicity.

The present invention provides a method of treating cancer in a patient comprising administering to the patient an effective amount of a compound which is 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol or a pharmaceutically acceptable salt thereof daily for at least 14 days of a 28-day cycle. Preferred is 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate. Preferred is that the compound or the salt thereof is administered daily for 14 days of a 28-day cycle. Preferred is that the compound or the salt thereof is administered daily for 21 days of a 28-day cycle. Preferred is that the compound or the salt thereof is administered at a dose between about 3.5 mg to about 100 mg daily. Preferred is that the compound or the salt thereof is administered at a dose of 5 mg once daily. Preferred is that the compound or the salt thereof is administered at a dose of 5 mg twice daily. Preferred is that the compound or the salt thereof is administered at a dose of 10 mg once daily. Preferred is that the compound or the salt thereof is administered at a dose of 10 mg twice daily. Preferred is that the compound or the salt thereof is administered at a dose of 20 mg once daily. Preferred is that the compound or the salt thereof is administered at a dose of 20 mg twice daily. Preferred is that the compound or the salt thereof is administered at a dose of 40 mg once daily. Preferred is that the compound or the salt thereof is administered at a dose of 25 mg once daily. Preferred is that the compound or the salt thereof is administered at a dose of 25 mg twice daily. Preferred is that the compound or the salt thereof is administered at a dose of 35 mg once daily. Preferred is that the compound or the salt thereof is administered at a dose of 35 mg twice daily. Preferred is that the compound or the salt thereof is administered at a dose of 70 mg once daily. Preferred is that the compound or the salt thereof is administered at a dose of 50 mg once daily. Preferred is that the compound or the salt thereof is administered at a dose of 50 mg twice daily. Preferred is that the compound or the salt thereof is administered at a dose of 100 mg once daily. Preferred is that the cancer is colorectal cancer, melanoma, hepatocellular carcinoma, renal cancer, brain cancer, pancreatic cancer, myelodysplastic syndrome, lung cancer, breast cancer, head and neck cancer, or gastric cancer. Preferred is that the cancer is colorectal cancer. Preferred is that the colorectal cancer is colon cancer. Preferred is that the colorectal cancer is rectal cancer. Preferred is that the cancer is melanoma. Preferred is that the cancer is hepatocellular carcinoma. Preferred is that the cancer is renal cancer. Preferred is that the cancer is brain cancer. Preferred is that the brain cancer is a glioma. Preferred is that the brain cancer is a glioma that has an IDH1 mutation. Preferred is that the brain cancer is a glioma that has an IDH2 mutation. Preferred is that the cancer is pancreatic cancer. Preferred is that the cancer is myelodysplastic syndrome. Preferred is that the cancer is lung cancer. Preferred is the cancer is breast cancer. Preferred is that the cancer is head and neck cancer. Preferred is that the cancer is gastric cancer.

This invention provides a compound which is 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol or a pharmaceutically acceptable salt thereof for use in the treatment of a cancer wherein the compound or the salt thereof is administered daily for at least 14 days of a 28-day cycle. Preferred is 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate. Preferred is that the compound or the salt thereof is administered daily for 14 days of a 28-day cycle. Preferred is that the compound or the salt thereof is administered daily for 21 days of a 28-day cycle. Preferred is that the compound or the salt thereof is administered at a dose between about 3.5 mg to about 100 mg daily. Preferred is that the compound or the salt thereof is administered at a dose of 5 mg once daily. Preferred is that the compound or the salt thereof is administered at a dose of 5 mg twice daily. Preferred is that the compound or the salt thereof is administered at a dose of 10 mg once daily. Preferred is that the compound or the salt thereof is administered at a dose of 10 mg twice daily. Preferred is that the compound or the salt thereof is administered at a dose of 20 mg once daily. Preferred is that the compound or the salt thereof is administered at a dose of 20 mg twice daily. Preferred is that the compound or the salt thereof is administered at a dose of 40 mg once daily. Preferred is that the compound or the salt thereof is administered at a dose of 25 mg once daily. Preferred is that the compound or the salt thereof is administered at a dose of 25 mg twice daily. Preferred is that the compound or the salt thereof is administered at a dose of 35 mg once daily. Preferred is that the compound or the salt thereof is administered at a dose of 35 mg twice daily. Preferred is that the compound or the salt thereof is administered at a dose of 70 mg once daily. Preferred is that the compound or the salt thereof is administered at a dose of 50 mg once daily. Preferred is that the compound or the salt thereof is administered at a dose of 50 mg twice daily. Preferred is that the compound or the salt thereof is administered at a dose of 100 mg once daily. Preferred is that the cancer is colorectal cancer, melanoma, hepatocellular carcinoma, renal cancer, brain cancer, pancreatic cancer, myelodysplastic syndrome, lung cancer, breast cancer, head and neck cancer, or gastric cancer. Preferred is that the cancer is colorectal cancer. Preferred is that the cancer is colorectal cancer and that the compound or the salt thereof is administered at a dose between about 3.5 mg to about 100 mg daily. Preferred is that the cancer is colorectal cancer and that the compound or the salt thereof is administered at a dose of 5 mg once daily. Preferred is that the cancer is colorectal cancer and that the compound or the salt thereof is administered at a dose of 5 mg twice daily. Preferred is that the cancer is colorectal cancer and that the compound or the salt thereof is administered at a dose of 10 mg once daily. Preferred is that the cancer is colorectal cancer and that the compound or the salt thereof is administered at a dose of 10 mg twice daily. Preferred is that the cancer is colorectal cancer and that the compound or the salt thereof is administered at a dose of 20 mg once daily. Preferred is that the cancer is colorectal cancer and that the compound or the salt thereof is administered at a dose of 20 mg twice daily. Preferred is that the cancer is colorectal cancer and that the compound or the salt thereof is administered at a dose of 40 mg once daily. Preferred is that the cancer is colorectal cancer and that the compound or the salt thereof is administered at a dose of 25 mg once daily. Preferred is that the cancer is colorectal cancer and that the compound or the salt thereof is administered at a dose of 25 mg twice daily. Preferred is that the cancer is colorectal cancer and that the compound or the salt thereof is administered at a dose of 35 mg once daily. Preferred is that the cancer is colorectal cancer and that the compound or the salt thereof is administered at a dose of 35 mg twice daily. Preferred is that the cancer is colorectal cancer and that the compound or the salt thereof is administered at a dose of 70 mg once daily. Preferred is that the cancer is colorectal cancer and that the compound or the salt thereof is administered at a dose of 50 mg once daily. Preferred is that the cancer is colorectal cancer and that the compound or the salt thereof is administered at a dose of 50 mg twice daily. Preferred is that the cancer is colorectal cancer and that the compound or the salt thereof is administered at a dose of 100 mg once daily. Preferred is that the colorectal cancer is colon cancer. Preferred is that the colorectal cancer is rectal cancer. Preferred is that the cancer is melanoma Preferred is that the cancer is melanoma and that the compound or the salt thereof is administered at a dose between about 3.5 mg to about 100 mg daily. Preferred is that the cancer is melanoma and that the compound or the salt thereof is administered at a dose of 5 mg once daily. Preferred is that the cancer is melanoma and that the compound or the salt thereof is administered at a dose of 5 mg twice daily. Preferred is that the cancer is melanoma and that the compound or the salt thereof is administered at a dose of 10 mg once daily. Preferred is that the cancer is melanoma and that the compound or the salt thereof is administered at a dose of 10 mg twice daily. Preferred is that the cancer is melanoma and that the compound or the salt thereof is administered at a dose of 20 mg once daily. Preferred is that the cancer is melanoma and that the compound or the salt thereof is administered at a dose of 20 mg twice daily. Preferred is that the cancer is melanoma and that the compound or the salt thereof is administered at a dose of 40 mg once daily. Preferred is that the cancer is melanoma and that the compound or the salt thereof is administered at a dose of 25 mg once daily. Preferred is that the cancer is melanoma and that the compound or the salt thereof is administered at a dose of 25 mg twice daily. Preferred is that the cancer is melanoma and that the compound or the salt thereof is administered at a dose of 35 mg once daily. Preferred is that the cancer is melanoma and that the compound or the salt thereof is administered at a dose of 35 mg twice daily. Preferred is that the cancer is melanoma and that the compound or the salt thereof is administered at a dose of 70 mg once daily. Preferred is that the cancer is melanoma and that the compound or the salt thereof is administered at a dose of 50 mg once daily. Preferred is that the cancer is melanoma and that the compound or the salt thereof is administered at a dose of 50 mg twice daily. Preferred is that the cancer is melanoma and that the compound or the salt thereof is administered at a dose of 100 mg once daily. Preferred is that the cancer is hepatocellular carcinoma. Preferred is that the cancer is hepatocellular carcinoma and that the compound or the salt thereof is administered at a dose between about 3.5 mg to about 100 mg daily. Preferred is that the cancer is hepatocellular carcinoma and that the compound or the salt thereof is administered at a dose of 5 mg once daily. Preferred is that the cancer is hepatocellular carcinoma and that the compound or the salt thereof is administered at a dose of 5 mg twice daily. Preferred is that the cancer is hepatocellular carcinoma and that the compound or the salt thereof is administered at a dose of 10 mg once daily. Preferred is that the cancer is hepatocellular carcinoma and that the compound or the salt thereof is administered at a dose of 10 mg twice daily. Preferred is that the cancer is hepatocellular carcinoma and that the compound or the salt thereof is administered at a dose of 20 mg once daily. Preferred is that the cancer is hepatocellular carcinoma and that the compound or the salt thereof is administered at a dose of 20 mg twice daily. Preferred is that the cancer is hepatocellular carcinoma and that the compound or the salt thereof is administered at a dose of 40 mg once daily. Preferred is that the cancer is hepatocellular carcinoma and that the compound or the salt thereof is administered at a dose of 25 mg once daily. Preferred is that the cancer is hepatocellular carcinoma and that the compound or the salt thereof is administered at a dose of 25 mg twice daily. Preferred is that the cancer is hepatocellular carcinoma and that the compound or the salt thereof is administered at a dose of 35 mg once daily. Preferred is that the cancer is hepatocellular carcinoma and that the compound or the salt thereof is administered at a dose of 35 mg twice daily. Preferred is that the cancer is hepatocellular carcinoma and that the compound or the salt thereof is administered at a dose of 70 mg once daily. Preferred is that the cancer is hepatocellular carcinoma and that the compound or the salt thereof is administered at a dose of 50 mg once daily. Preferred is that the cancer is hepatocellular carcinoma and that the compound or the salt thereof is administered at a dose of 50 mg twice daily. Preferred is that the cancer is hepatocellular carcinoma and that the compound or the salt thereof is administered at a dose of 100 mg once daily. Preferred is that the cancer is renal cancer. Preferred is that the cancer is renal cancer and that the compound or the salt thereof is administered at a dose between about 3.5 mg to about 100 mg daily. Preferred is that the cancer is renal cancer and that the compound or the salt thereof is administered at a dose of 5 mg once daily. Preferred is that the cancer is renal cancer and that the compound or the salt thereof is administered at a dose of 5 mg twice daily. Preferred is that the cancer is renal cancer and that the compound or the salt thereof is administered at a dose of 10 mg once daily. Preferred is that the cancer is renal cancer and that the compound or the salt thereof is administered at a dose of 10 mg twice daily. Preferred is that the cancer is renal cancer and that the compound or the salt thereof is administered at a dose of 20 mg once daily. Preferred is that the cancer is renal cancer and that the compound or the salt thereof is administered at a dose of 20 mg twice daily. Preferred is that the cancer is renal cancer and that the compound or the salt thereof is administered at a dose of 40 mg once daily. Preferred is that the cancer is renal cancer and that the compound or the salt thereof is administered at a dose of 25 mg once daily. Preferred is that the cancer is renal cancer and that the compound or the salt thereof is administered at a dose of 25 mg twice daily. Preferred is that the cancer is renal cancer and that the compound or the salt thereof is administered at a dose of 35 mg once daily. Preferred is that the cancer is renal cancer and that the compound or the salt thereof is administered at a dose of 35 mg twice daily. Preferred is that the cancer is renal cancer and that the compound or the salt thereof is administered at a dose of 70 mg once daily. Preferred is that the cancer is renal cancer and that the compound or the salt thereof is administered at a dose of 50 mg once daily. Preferred is that the cancer is renal cancer and that the compound or the salt thereof is administered at a dose of 50 mg twice daily. Preferred is that the cancer is renal cancer and that the compound or the salt thereof is administered at a dose of 100 mg once daily. Preferred is that the cancer is brain cancer. Preferred is that the cancer is brain cancer and that the compound or the salt thereof is administered at a dose between about 3.5 mg to about 100 mg daily. Preferred is that the cancer is brain cancer and that the compound or the salt thereof is administered at a dose of 5 mg once daily. Preferred is that the cancer is brain cancer and that the compound or the salt thereof is administered at a dose of 5 mg twice daily. Preferred is that the cancer is brain cancer and that the compound or the salt thereof is administered at a dose of 10 mg once daily. Preferred is that the cancer is brain cancer and that the compound or the salt thereof is administered at a dose of 10 mg twice daily. Preferred is that the cancer is brain cancer and that the compound or the salt thereof is administered at a dose of 20 mg once daily. Preferred is that the cancer is brain cancer and that the compound or the salt thereof is administered at a dose of 20 mg twice daily. Preferred is that the cancer is brain cancer and that the compound or the salt thereof is administered at a dose of 40 mg once daily. Preferred is that the cancer is brain cancer and that the compound or the salt thereof is administered at a dose of 25 mg once daily. Preferred is that the cancer is brain cancer and that the compound or the salt thereof is administered at a dose of 25 mg twice daily. Preferred is that the cancer is brain cancer and that the compound or the salt thereof is administered at a dose of 35 mg once daily. Preferred is that the cancer is brain cancer and that the compound or the salt thereof is administered at a dose of 35 mg twice daily. Preferred is that the cancer is brain cancer and that the compound or the salt thereof is administered at a dose of 70 mg once daily. Preferred is that the cancer is brain cancer and that the compound or the salt thereof is administered at a dose of 50 mg once daily. Preferred is that the cancer is brain cancer and that the compound or the salt thereof is administered at a dose of 50 mg twice daily. Preferred is that the cancer is brain cancer and that the compound or the salt thereof is administered at a dose of 100 mg once daily. Preferred is that the brain cancer is a glioma. Preferred is that the brain cancer is a glioma that has an IDH1 mutation. Preferred is that the brain cancer is a glioma that has an IDH2 mutation. Preferred is that the cancer is pancreatic cancer. Preferred is that the cancer is pancreatic cancer and that the compound or the salt thereof is administered at a dose between about 3.5 mg to about 100 mg daily. Preferred is that the cancer is pancreatic cancer and that the compound or the salt thereof is administered at a dose of 5 mg once daily. Preferred is that the cancer is pancreatic cancer and that the compound or the salt thereof is administered at a dose of 5 mg twice daily. Preferred is that the cancer is pancreatic cancer and that the compound or the salt thereof is administered at a dose of 10 mg once daily. Preferred is that the cancer is pancreatic cancer and that the compound or the salt thereof is administered at a dose of 10 mg twice daily. Preferred is that the cancer is pancreatic cancer and that the compound or the salt thereof is administered at a dose of 20 mg once daily. Preferred is that the cancer is pancreatic cancer and that the compound or the salt thereof is administered at a dose of 20 mg twice daily. Preferred is that the cancer is pancreatic cancer and that the compound or the salt thereof is administered at a dose of 40 mg once daily. Preferred is that the cancer is pancreatic cancer and that the compound or the salt thereof is administered at a dose of 25 mg once daily. Preferred is that the cancer is pancreatic cancer and that the compound or the salt thereof is administered at a dose of 25 mg twice daily. Preferred is that the cancer is pancreatic cancer and that the compound or the salt thereof is administered at a dose of 35 mg once daily. Preferred is that the cancer is pancreatic cancer and that the compound or the salt thereof is administered at a dose of 35 mg twice daily. Preferred is that the cancer is pancreatic cancer and that the compound or the salt thereof is administered at a dose of 70 mg once daily. Preferred is that the cancer is pancreatic cancer and that the compound or the salt thereof is administered at a dose of 50 mg once daily. Preferred is that the cancer is pancreatic cancer and that the compound or the salt thereof is administered at a dose of 50 mg twice daily. Preferred is that the cancer is pancreatic cancer and that the compound or the salt thereof is administered at a dose of 100 mg once daily. Preferred is that the cancer is myelodysplastic syndrome. Preferred is that the cancer is myelodysplastic syndrome and that the compound or the salt thereof is administered at a dose between about 3.5 mg to about 100 mg daily. Preferred is that the cancer is myelodysplastic syndrome and that the compound or the salt thereof is administered at a dose of 5 mg once daily. Preferred is that the cancer is myelodysplastic syndrome and that the compound or the salt thereof is administered at a dose of 5 mg twice daily. Preferred is that the cancer is myelodysplastic syndrome and that the compound or the salt thereof is administered at a dose of 10 mg once daily. Preferred is that the cancer is myelodysplastic syndrome and that the compound or the salt thereof is administered at a dose of 10 mg twice daily. Preferred is that the cancer is myelodysplastic syndrome and that the compound or the salt thereof is administered at a dose of 20 mg once daily. Preferred is that the cancer is myelodysplastic syndrome and that the compound or the salt thereof is administered at a dose of 20 mg twice daily. Preferred is that the cancer is myelodysplastic syndrome and that the compound or the salt thereof is administered at a dose of 40 mg once daily. Preferred is that the cancer is myelodysplastic syndrome and that the compound or the salt thereof is administered at a dose of 25 mg once daily. Preferred is that the cancer is myelodysplastic syndrome and that the compound or the salt thereof is administered at a dose of 25 mg twice daily. Preferred is that the cancer is myelodysplastic syndrome and that the compound or the salt thereof is administered at a dose of 35 mg once daily. Preferred is that the cancer is myelodysplastic syndrome and that the compound or the salt thereof is administered at a dose of 35 mg twice daily. Preferred is that the cancer is myelodysplastic syndrome and that the compound or the salt thereof is administered at a dose of 70 mg once daily. Preferred is that the cancer is myelodysplastic syndrome and that the compound or the salt thereof is administered at a dose of 50 mg once daily. Preferred is that the cancer is myelodysplastic syndrome and that the compound or the salt thereof is administered at a dose of 50 mg twice daily. Preferred is that the cancer is myelodysplastic syndrome and that the compound or the salt thereof is administered at a dose of 100 mg once daily. Preferred is that the cancer is lung cancer. Preferred is that the cancer is lung cancer and that the compound or the salt thereof is administered at a dose between about 3.5 mg to about 100 mg daily. Preferred is that the cancer is lung cancer and that the compound or the salt thereof is administered at a dose of 5 mg once daily. Preferred is that the cancer is lung cancer and that the compound or the salt thereof is administered at a dose of 5 mg twice daily. Preferred is that the cancer is lung cancer and that the compound or the salt thereof is administered at a dose of 10 mg once daily. Preferred is that the cancer is lung cancer and that the compound or the salt thereof is administered at a dose of 10 mg twice daily. Preferred is that the cancer is lung cancer and that the compound or the salt thereof is administered at a dose of 20 mg once daily. Preferred is that the cancer is lung cancer and that the compound or the salt thereof is administered at a dose of 20 mg twice daily. Preferred is that the cancer is lung cancer and that the compound or the salt thereof is administered at a dose of 40 mg once daily. Preferred is that the cancer is lung cancer and that the compound or the salt thereof is administered at a dose of 25 mg once daily. Preferred is that the cancer is lung cancer and that the compound or the salt thereof is administered at a dose of 25 mg twice daily. Preferred is that the cancer is lung cancer and that the compound or the salt thereof is administered at a dose of 35 mg once daily. Preferred is that the cancer is lung cancer and that the compound or the salt thereof is administered at a dose of 35 mg twice daily. Preferred is that the cancer is lung cancer and that the compound or the salt thereof is administered at a dose of 70 mg once daily. Preferred is that the cancer is lung cancer and that the compound or the salt thereof is administered at a dose of 50 mg once daily. Preferred is that the cancer is lung cancer and that the compound or the salt thereof is administered at a dose of 50 mg twice daily. Preferred is that the cancer is lung cancer and that the compound or the salt thereof is administered at a dose of 100 mg once daily. Preferred is the cancer is breast cancer. Preferred is that the cancer is breast cancer and that the compound or the salt thereof is administered at a dose between about 3.5 mg to about 100 mg daily. Preferred is that the cancer is breast cancer and that the compound or the salt thereof is administered at a dose of 5 mg once daily. Preferred is that the cancer is breast cancer and that the compound or the salt thereof is administered at a dose of 5 mg twice daily. Preferred is that the cancer is breast cancer and that the compound or the salt thereof is administered at a dose of 10 mg once daily. Preferred is that the cancer is breast cancer and that the compound or the salt thereof is administered at a dose of 10 mg twice daily. Preferred is that the cancer is breast cancer and that the compound or the salt thereof is administered at a dose of 20 mg once daily. Preferred is that the cancer is breast cancer and that the compound or the salt thereof is administered at a dose of 20 mg twice daily. Preferred is that the cancer is breast cancer and that the compound or the salt thereof is administered at a dose of 40 mg once daily. Preferred is that the cancer is breast cancer and that the compound or the salt thereof is administered at a dose of 25 mg once daily. Preferred is that the cancer is breast cancer and that the compound or the salt thereof is administered at a dose of 25 mg twice daily. Preferred is that the cancer is breast cancer and that the compound or the salt thereof is administered at a dose of 35 mg once daily. Preferred is that the cancer is breast cancer and that the compound or the salt thereof is administered at a dose of 35 mg twice daily. Preferred is that the cancer is breast cancer and that the compound or the salt thereof is administered at a dose of 70 mg once daily. Preferred is that the cancer is breast cancer and that the compound or the salt thereof is administered at a dose of 50 mg once daily. Preferred is that the cancer is breast cancer and that the compound or the salt thereof is administered at a dose of 50 mg twice daily. Preferred is that the cancer is breast cancer and that the compound or the salt thereof is administered at a dose of 100 mg once daily. Preferred is that the cancer is head and neck cancer. Preferred is that the cancer is head and neck cancer and that the compound or the salt thereof is administered at a dose between about 3.5 mg to about 100 mg daily. Preferred is that the cancer is head and neck cancer and that the compound or the salt thereof is administered at a dose of 5 mg once daily. Preferred is that the cancer is head and neck cancer and that the compound or the salt thereof is administered at a dose of 5 mg twice daily. Preferred is that the cancer is head and neck cancer and that the compound or the salt thereof is administered at a dose of 10 mg once daily. Preferred is that the cancer is head and neck cancer and that the compound or the salt thereof is administered at a dose of 10 mg twice daily. Preferred is that the cancer is head and neck cancer and that the compound or the salt thereof is administered at a dose of 20 mg once daily. Preferred is that the cancer is head and neck cancer and that the compound or the salt thereof is administered at a dose of 20 mg twice daily. Preferred is that the cancer is head and neck cancer and that the compound or the salt thereof is administered at a dose of 40 mg once daily. Preferred is that the cancer is head and neck cancer and that the compound or the salt thereof is administered at a dose of 25 mg once daily. Preferred is that the cancer is head and neck cancer and that the compound or the salt thereof is administered at a dose of 25 mg twice daily. Preferred is that the cancer is head and neck cancer and that the compound or the salt thereof is administered at a dose of 35 mg once daily. Preferred is that the cancer is head and neck cancer and that the compound or the salt thereof is administered at a dose of 35 mg twice daily. Preferred is that the cancer is head and neck cancer and that the compound or the salt thereof is administered at a dose of 70 mg once daily. Preferred is that the cancer is head and neck cancer and that the compound or the salt thereof is administered at a dose of 50 mg once daily. Preferred is that the cancer is head and neck cancer and that the compound or the salt thereof is administered at a dose of 50 mg twice daily. Preferred is that the cancer is head and neck cancer and that the compound or the salt thereof is administered at a dose of 100 mg once daily. Preferred is that the cancer is gastric cancer. Preferred is that the cancer is gastric cancer and that the compound or the salt thereof is administered at a dose between about 3.5 mg to about 100 mg daily. Preferred is that the cancer is gastric cancer and that the compound or the salt thereof is administered at a dose of 5 mg once daily. Preferred is that the cancer is gastric cancer and that the compound or the salt thereof is administered at a dose of 5 mg twice daily. Preferred is that the cancer is gastric cancer and that the compound or the salt thereof is administered at a dose of 10 mg once daily. Preferred is that the cancer is gastric cancer and that the compound or the salt thereof is administered at a dose of 10 mg twice daily. Preferred is that the cancer is gastric cancer and that the compound or the salt thereof is administered at a dose of 20 mg once daily. Preferred is that the cancer is gastric cancer and that the compound or the salt thereof is administered at a dose of 20 mg twice daily. Preferred is that the cancer is gastric cancer and that the compound or the salt thereof is administered at a dose of 40 mg once daily. Preferred is that the cancer is gastric cancer and that the compound or the salt thereof is administered at a dose of 25 mg once daily. Preferred is that the cancer is gastric cancer and that the compound or the salt thereof is administered at a dose of 25 mg twice daily. Preferred is that the cancer is gastric cancer and that the compound or the salt thereof is administered at a dose of 35 mg once daily. Preferred is that the cancer is gastric cancer and that the compound or the salt thereof is administered at a dose of 35 mg twice daily. Preferred is that the cancer is gastric cancer and that the compound or the salt thereof is administered at a dose of 70 mg once daily. Preferred is that the cancer is gastric cancer and that the compound or the salt thereof is administered at a dose of 50 mg once daily. Preferred is that the cancer is gastric cancer and that the compound or the salt thereof is administered at a dose of 50 mg twice daily. Preferred is that the cancer is gastric cancer and that the compound or the salt thereof is administered at a dose of 100 mg once daily.

Furthermore, this invention provides the use of a compound which is 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol, or a pharmaceutically acceptable salt thereof, for the manufacture of a medicament for the treatment of cancer wherein the compound or the salt thereof is administered daily for at least 14 days of a 28-day cycle. Preferred is 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate. Preferred is that the compound or the salt thereof is administered daily for 14 days of a 28-day cycle. Preferred is that the compound or the salt thereof is administered daily for 21 days of a 28-day cycle. Preferred is that the compound or the salt thereof is administered at a dose between about 3.5 mg to about 100 mg daily. Preferred is that the compound or the salt thereof is administered at a dose of 5 mg once daily. Preferred is that the compound or the salt thereof is administered at a dose of 5 mg twice daily. Preferred is that the compound or the salt thereof is administered at a dose of 10 mg once daily. Preferred is that the compound or the salt thereof is administered at a dose of 10 mg twice daily. Preferred is that the compound or the salt thereof is administered at a dose of 20 mg once daily. Preferred is that the compound or the salt thereof is administered at a dose of 20 mg twice daily. Preferred is that the compound or the salt thereof is administered at a dose of 40 mg once daily. Preferred is that the compound or the salt thereof is administered at a dose of 25 mg once daily. Preferred is that the compound or the salt thereof is administered at a dose of 25 mg twice daily. Preferred is that the compound or the salt thereof is administered at a dose of 35 mg once daily. Preferred is that the compound or the salt thereof is administered at a dose of 35 mg twice daily. Preferred is that the compound or the salt thereof is administered at a dose of 70 mg once daily. Preferred is that the compound or the salt thereof is administered at a dose of 50 mg once daily. Preferred is that the compound or the salt thereof is administered at a dose of 50 mg twice daily. Preferred is that the compound or the salt thereof is administered at a dose of 100 mg once daily. Preferred is that the cancer is colorectal cancer, melanoma, hepatocellular carcinoma, renal cancer, brain cancer, pancreatic cancer, myelodysplastic syndrome, lung cancer, breast cancer, head and neck cancer, or gastric cancer. Preferred is that the cancer is colorectal cancer. Preferred is that the cancer is colorectal cancer and that the compound or the salt thereof is administered at a dose between about 3.5 mg to about 100 mg daily. Preferred is that the cancer is colorectal cancer and that the compound or the salt thereof is administered at a dose of 5 mg once daily. Preferred is that the cancer is colorectal cancer and that the compound or the salt thereof is administered at a dose of 5 mg twice daily. Preferred is that the cancer is colorectal cancer and that the compound or the salt thereof is administered at a dose of 10 mg once daily. Preferred is that the cancer is colorectal cancer and that the compound or the salt thereof is administered at a dose of 10 mg twice daily. Preferred is that the cancer is colorectal cancer and that the compound or the salt thereof is administered at a dose of 20 mg once daily. Preferred is that the cancer is colorectal cancer and that the compound or the salt thereof is administered at a dose of 20 mg twice daily. Preferred is that the cancer is colorectal cancer and that the compound or the salt thereof is administered at a dose of 40 mg once daily. Preferred is that the cancer is colorectal cancer and that the compound or the salt thereof is administered at a dose of 25 mg once daily. Preferred is that the cancer is colorectal cancer and that the compound or the salt thereof is administered at a dose of 25 mg twice daily. Preferred is that the cancer is colorectal cancer and that the compound or the salt thereof is administered at a dose of 35 mg once daily. Preferred is that the cancer is colorectal cancer and that the compound or the salt thereof is administered at a dose of 35 mg twice daily. Preferred is that the cancer is colorectal cancer and that the compound or the salt thereof is administered at a dose of 70 mg once daily. Preferred is that the cancer is colorectal cancer and that the compound or the salt thereof is administered at a dose of 50 mg once daily. Preferred is that the cancer is colorectal cancer and that the compound or the salt thereof is administered at a dose of 50 mg twice daily. Preferred is that the cancer is colorectal cancer and that the compound or the salt thereof is administered at a dose of 100 mg once daily. Preferred is that the colorectal cancer is colon cancer. Preferred is that the colorectal cancer is rectal cancer. Preferred is that the cancer is melanoma. Preferred is that the cancer is melanoma and that the compound or the salt thereof is administered at a dose between about 3.5 mg to about 100 mg daily. Preferred is that the cancer is melanoma and that the compound or the salt thereof is administered at a dose of 5 mg once daily. Preferred is that the cancer is melanoma and that the compound or the salt thereof is administered at a dose of 5 mg twice daily. Preferred is that the cancer is melanoma and that the compound or the salt thereof is administered at a dose of 10 mg once daily. Preferred is that the cancer is melanoma and that the compound or the salt thereof is administered at a dose of 10 mg twice daily. Preferred is that the cancer is melanoma and that the compound or the salt thereof is administered at a dose of 20 mg once daily. Preferred is that the cancer is melanoma and that the compound or the salt thereof is administered at a dose of 20 mg twice daily. Preferred is that the cancer is melanoma and that the compound or the salt thereof is administered at a dose of 40 mg once daily. Preferred is that the cancer is melanoma and that the compound or the salt thereof is administered at a dose of 25 mg once daily. Preferred is that the cancer is melanoma and that the compound or the salt thereof is administered at a dose of 25 mg twice daily. Preferred is that the cancer is melanoma and that the compound or the salt thereof is administered at a dose of 35 mg once daily. Preferred is that the cancer is melanoma and that the compound or the salt thereof is administered at a dose of 35 mg twice daily. Preferred is that the cancer is melanoma and that the compound or the salt thereof is administered at a dose of 70 mg once daily. Preferred is that the cancer is melanoma and that the compound or the salt thereof is administered at a dose of 50 mg once daily. Preferred is that the cancer is melanoma and that the compound or the salt thereof is administered at a dose of 50 mg twice daily. Preferred is that the cancer is melanoma and that the compound or the salt thereof is administered at a dose of 100 mg once daily. Preferred is that the cancer is hepatocellular carcinoma. Preferred is that the cancer is hepatocellular carcinoma and that the compound or the salt thereof is administered at a dose between about 3.5 mg to about 100 mg daily. Preferred is that the cancer is hepatocellular carcinoma and that the compound or the salt thereof is administered at a dose of 5 mg once daily. Preferred is that the cancer is hepatocellular carcinoma and that the compound or the salt thereof is administered at a dose of 5 mg twice daily. Preferred is that the cancer is hepatocellular carcinoma and that the compound or the salt thereof is administered at a dose of 10 mg once daily. Preferred is that the cancer is hepatocellular carcinoma and that the compound or the salt thereof is administered at a dose of 10 mg twice daily. Preferred is that the cancer is hepatocellular carcinoma and that the compound or the salt thereof is administered at a dose of 20 mg once daily. Preferred is that the cancer is hepatocellular carcinoma and that the compound or the salt thereof is administered at a dose of 20 mg twice daily. Preferred is that the cancer is hepatocellular carcinoma and that the compound or the salt thereof is administered at a dose of 40 mg once daily. Preferred is that the cancer is hepatocellular carcinoma and that the compound or the salt thereof is administered at a dose of 25 mg once daily. Preferred is that the cancer is hepatocellular carcinoma and that the compound or the salt thereof is administered at a dose of 25 mg twice daily. Preferred is that the cancer is hepatocellular carcinoma and that the compound or the salt thereof is administered at a dose of 35 mg once daily. Preferred is that the cancer is hepatocellular carcinoma and that the compound or the salt thereof is administered at a dose of 35 mg twice daily. Preferred is that the cancer is hepatocellular carcinoma and that the compound or the salt thereof is administered at a dose of 70 mg once daily. Preferred is that the cancer is hepatocellular carcinoma and that the compound or the salt thereof is administered at a dose of 50 mg once daily. Preferred is that the cancer is hepatocellular carcinoma and that the compound or the salt thereof is administered at a dose of 50 mg twice daily. Preferred is that the cancer is hepatocellular carcinoma and that the compound or the salt thereof is administered at a dose of 100 mg once daily. Preferred is that the cancer is renal cancer. Preferred is that the cancer is renal cancer and that the compound or the salt thereof is administered at a dose between about 3.5 mg to about 100 mg daily. Preferred is that the cancer is renal cancer and that the compound or the salt thereof is administered at a dose of 5 mg once daily. Preferred is that the cancer is renal cancer and that the compound or the salt thereof is administered at a dose of 5 mg twice daily. Preferred is that the cancer is renal cancer and that the compound or the salt thereof is administered at a dose of 10 mg once daily. Preferred is that the cancer is renal cancer and that the compound or the salt thereof is administered at a dose of 10 mg twice daily. Preferred is that the cancer is renal cancer and that the compound or the salt thereof is administered at a dose of 20 mg once daily. Preferred is that the cancer is renal cancer and that the compound or the salt thereof is administered at a dose of 20 mg twice daily. Preferred is that the cancer is renal cancer and that the compound or the salt thereof is administered at a dose of 40 mg once daily. Preferred is that the cancer is renal cancer and that the compound or the salt thereof is administered at a dose of 25 mg once daily. Preferred is that the cancer is renal cancer and that the compound or the salt thereof is administered at a dose of 25 mg twice daily. Preferred is that the cancer is renal cancer and that the compound or the salt thereof is administered at a dose of 35 mg once daily. Preferred is that the cancer is renal cancer and that the compound or the salt thereof is administered at a dose of 35 mg twice daily. Preferred is that the cancer is renal cancer and that the compound or the salt thereof is administered at a dose of 70 mg once daily. Preferred is that the cancer is renal cancer and that the compound or the salt thereof is administered at a dose of 50 mg once daily. Preferred is that the cancer is renal cancer and that the compound or the salt thereof is administered at a dose of 50 mg twice daily. Preferred is that the cancer is renal cancer and that the compound or the salt thereof is administered at a dose of 100 mg once daily. Preferred is that the cancer is brain cancer. Preferred is that the cancer is brain cancer and that the compound or the salt thereof is administered at a dose between about 3.5 mg to about 100 mg daily. Preferred is that the cancer is brain cancer and that the compound or the salt thereof is administered at a dose of 5 mg once daily. Preferred is that the cancer is brain cancer and that the compound or the salt thereof is administered at a dose of 5 mg twice daily. Preferred is that the cancer is brain cancer and that the compound or the salt thereof is administered at a dose of 10 mg once daily. Preferred is that the cancer is brain cancer and that the compound or the salt thereof is administered at a dose of 10 mg twice daily. Preferred is that the cancer is brain cancer and that the compound or the salt thereof is administered at a dose of 20 mg once daily. Preferred is that the cancer is brain cancer and that the compound or the salt thereof is administered at a dose of 20 mg twice daily. Preferred is that the cancer is brain cancer and that the compound or the salt thereof is administered at a dose of 40 mg once daily. Preferred is that the cancer is brain cancer and that the compound or the salt thereof is administered at a dose of 25 mg once daily. Preferred is that the cancer is brain cancer and that the compound or the salt thereof is administered at a dose of 25 mg twice daily. Preferred is that the cancer is brain cancer and that the compound or the salt thereof is administered at a dose of 35 mg once daily. Preferred is that the cancer is brain cancer and that the compound or the salt thereof is administered at a dose of 35 mg twice daily. Preferred is that the cancer is brain cancer and that the compound or the salt thereof is administered at a dose of 70 mg once daily. Preferred is that the cancer is brain cancer and that the compound or the salt thereof is administered at a dose of 50 mg once daily. Preferred is that the cancer is brain cancer and that the compound or the salt thereof is administered at a dose of 50 mg twice daily. Preferred is that the cancer is brain cancer and that the compound or the salt thereof is administered at a dose of 100 mg once daily. Preferred is that the brain cancer is a glioma. Preferred is that the brain cancer is a glioma that has an IDH1 mutation. Preferred is that the brain cancer is a glioma that has an IDH2 mutation. Preferred is that the cancer is pancreatic cancer. Preferred is that the cancer is pancreatic cancer and that the compound or the salt thereof is administered at a dose between about 3.5 mg to about 100 mg daily. Preferred is that the cancer is pancreatic cancer and that the compound or the salt thereof is administered at a dose of 5 mg once daily. Preferred is that the cancer is pancreatic cancer and that the compound or the salt thereof is administered at a dose of 5 mg twice daily. Preferred is that the cancer is pancreatic cancer and that the compound or the salt thereof is administered at a dose of 10 mg once daily. Preferred is that the cancer is pancreatic cancer and that the compound or the salt thereof is administered at a dose of 10 mg twice daily. Preferred is that the cancer is pancreatic cancer and that the compound or the salt thereof is administered at a dose of 20 mg once daily. Preferred is that the cancer is pancreatic cancer and that the compound or the salt thereof is administered at a dose of 20 mg twice daily. Preferred is that the cancer is pancreatic cancer and that the compound or the salt thereof is administered at a dose of 40 mg once daily. Preferred is that the cancer is pancreatic cancer and that the compound or the salt thereof is administered at a dose of 25 mg once daily. Preferred is that the cancer is pancreatic cancer and that the compound or the salt thereof is administered at a dose of 25 mg twice daily. Preferred is that the cancer is pancreatic cancer and that the compound or the salt thereof is administered at a dose of 35 mg once daily. Preferred is that the cancer is pancreatic cancer and that the compound or the salt thereof is administered at a dose of 35 mg twice daily. Preferred is that the cancer is pancreatic cancer and that the compound or the salt thereof is administered at a dose of 70 mg once daily. Preferred is that the cancer is pancreatic cancer and that the compound or the salt thereof is administered at a dose of 50 mg once daily. Preferred is that the cancer is pancreatic cancer and that the compound or the salt thereof is administered at a dose of 50 mg twice daily. Preferred is that the cancer is pancreatic cancer and that the compound or the salt thereof is administered at a dose of 100 mg once daily. Preferred is that the cancer is myelodysplastic syndrome. Preferred is that the cancer is myelodysplastic syndrome and that the compound or the salt thereof is administered at a dose between about 3.5 mg to about 100 mg daily. Preferred is that the cancer is myelodysplastic syndrome and that the compound or the salt thereof is administered at a dose of 5 mg once daily. Preferred is that the cancer is myelodysplastic syndrome and that the compound or the salt thereof is administered at a dose of 5 mg twice daily. Preferred is that the cancer is myelodysplastic syndrome and that the compound or the salt thereof is administered at a dose of 10 mg once daily. Preferred is that the cancer is myelodysplastic syndrome and that the compound or the salt thereof is administered at a dose of 10 mg twice daily. Preferred is that the cancer is myelodysplastic syndrome and that the compound or the salt thereof is administered at a dose of 20 mg once daily. Preferred is that the cancer is myelodysplastic syndrome and that the compound or the salt thereof is administered at a dose of 20 mg twice daily. Preferred is that the cancer is myelodysplastic syndrome and that the compound or the salt thereof is administered at a dose of 40 mg once daily. Preferred is that the cancer is myelodysplastic syndrome and that the compound or the salt thereof is administered at a dose of 25 mg once daily. Preferred is that the cancer is myelodysplastic syndrome and that the compound or the salt thereof is administered at a dose of 25 mg twice daily. Preferred is that the cancer is myelodysplastic syndrome and that the compound or the salt thereof is administered at a dose of 35 mg once daily. Preferred is that the cancer is myelodysplastic syndrome and that the compound or the salt thereof is administered at a dose of 35 mg twice daily. Preferred is that the cancer is myelodysplastic syndrome and that the compound or the salt thereof is administered at a dose of 70 mg once daily. Preferred is that the cancer is myelodysplastic syndrome and that the compound or the salt thereof is administered at a dose of 50 mg once daily. Preferred is that the cancer is myelodysplastic syndrome and that the compound or the salt thereof is administered at a dose of 50 mg twice daily. Preferred is that the cancer is myelodysplastic syndrome and that the compound or the salt thereof is administered at a dose of 100 mg once daily. Preferred is that the cancer is lung cancer. Preferred is that the cancer is lung cancer and that the compound or the salt thereof is administered at a dose between about 3.5 mg to about 100 mg daily. Preferred is that the cancer is lung cancer and that the compound or the salt thereof is administered at a dose of 5 mg once daily. Preferred is that the cancer is lung cancer and that the compound or the salt thereof is administered at a dose of 5 mg twice daily. Preferred is that the cancer is lung cancer and that the compound or the salt thereof is administered at a dose of 10 mg once daily. Preferred is that the cancer is lung cancer and that the compound or the salt thereof is administered at a dose of 10 mg twice daily. Preferred is that the cancer is lung cancer and that the compound or the salt thereof is administered at a dose of 20 mg once daily. Preferred is that the cancer is lung cancer and that the compound or the salt thereof is administered at a dose of 20 mg twice daily. Preferred is that the cancer is lung cancer and that the compound or the salt thereof is administered at a dose of 40 mg once daily. Preferred is that the cancer is lung cancer and that the compound or the salt thereof is administered at a dose of 25 mg once daily. Preferred is that the cancer is lung cancer and that the compound or the salt thereof is administered at a dose of 25 mg twice daily. Preferred is that the cancer is lung cancer and that the compound or the salt thereof is administered at a dose of 35 mg once daily. Preferred is that the cancer is lung cancer and that the compound or the salt thereof is administered at a dose of 35 mg twice daily. Preferred is that the cancer is lung cancer and that the compound or the salt thereof is administered at a dose of 70 mg once daily. Preferred is that the cancer is lung cancer and that the compound or the salt thereof is administered at a dose of 50 mg once daily. Preferred is that the cancer is lung cancer and that the compound or the salt thereof is administered at a dose of 50 mg twice daily. Preferred is that the cancer is lung cancer and that the compound or the salt thereof is administered at a dose of 100 mg once daily. Preferred is the cancer is breast cancer. Preferred is that the cancer is breast cancer and that the compound or the salt thereof is administered at a dose between about 3.5 mg to about 100 mg daily. Preferred is that the cancer is breast cancer and that the compound or the salt thereof is administered at a dose of 5 mg once daily. Preferred is that the cancer is breast cancer and that the compound or the salt thereof is administered at a dose of 5 mg twice daily. Preferred is that the cancer is breast cancer and that the compound or the salt thereof is administered at a dose of 10 mg once daily. Preferred is that the cancer is breast cancer and that the compound or the salt thereof is administered at a dose of 10 mg twice daily. Preferred is that the cancer is breast cancer and that the compound or the salt thereof is administered at a dose of 20 mg once daily. Preferred is that the cancer is breast cancer and that the compound or the salt thereof is administered at a dose of 20 mg twice daily. Preferred is that the cancer is breast cancer and that the compound or the salt thereof is administered at a dose of 40 mg once daily. Preferred is that the cancer is breast cancer and that the compound or the salt thereof is administered at a dose of 25 mg once daily. Preferred is that the cancer is breast cancer and that the compound or the salt thereof is administered at a dose of 25 mg twice daily. Preferred is that the cancer is breast cancer and that the compound or the salt thereof is administered at a dose of 35 mg once daily. Preferred is that the cancer is breast cancer and that the compound or the salt thereof is administered at a dose of 35 mg twice daily. Preferred is that the cancer is breast cancer and that the compound or the salt thereof is administered at a dose of 70 mg once daily. Preferred is that the cancer is breast cancer and that the compound or the salt thereof is administered at a dose of 50 mg once daily. Preferred is that the cancer is breast cancer and that the compound or the salt thereof is administered at a dose of 50 mg twice daily. Preferred is that the cancer is breast cancer and that the compound or the salt thereof is administered at a dose of 100 mg once daily. Preferred is that the cancer is head and neck cancer. Preferred is that the cancer is head and neck cancer and that the compound or the salt thereof is administered at a dose between about 3.5 mg to about 100 mg daily. Preferred is that the cancer is head and neck cancer and that the compound or the salt thereof is administered at a dose of 5 mg once daily. Preferred is that the cancer is head and neck cancer and that the compound or the salt thereof is administered at a dose of 5 mg twice daily. Preferred is that the cancer is head and neck cancer and that the compound or the salt thereof is administered at a dose of 10 mg once daily. Preferred is that the cancer is head and neck cancer and that the compound or the salt thereof is administered at a dose of 10 mg twice daily. Preferred is that the cancer is head and neck cancer and that the compound or the salt thereof is administered at a dose of 20 mg once daily. Preferred is that the cancer is head and neck cancer and that the compound or the salt thereof is administered at a dose of 20 mg twice daily. Preferred is that the cancer is head and neck cancer and that the compound or the salt thereof is administered at a dose of 40 mg once daily. Preferred is that the cancer is head and neck cancer and that the compound or the salt thereof is administered at a dose of 25 mg once daily. Preferred is that the cancer is head and neck cancer and that the compound or the salt thereof is administered at a dose of 25 mg twice daily. Preferred is that the cancer is head and neck cancer and that the compound or the salt thereof is administered at a dose of 35 mg once daily. Preferred is that the cancer is head and neck cancer and that the compound or the salt thereof is administered at a dose of 35 mg twice daily. Preferred is that the cancer is head and neck cancer and that the compound or the salt thereof is administered at a dose of 70 mg once daily. Preferred is that the cancer is head and neck cancer and that the compound or the salt thereof is administered at a dose of 50 mg once daily. Preferred is that the cancer is head and neck cancer and that the compound or the salt thereof is administered at a dose of 50 mg twice daily. Preferred is that the cancer is head and neck cancer and that the compound or the salt thereof is administered at a dose of 100 mg once daily. Preferred is that the cancer is gastric cancer. Preferred is that the cancer is gastric cancer and that the compound or the salt thereof is administered at a dose between about 3.5 mg to about 100 mg daily. Preferred is that the cancer is gastric cancer and that the compound or the salt thereof is administered at a dose of 5 mg once daily. Preferred is that the cancer is gastric cancer and that the compound or the salt thereof is administered at a dose of 5 mg twice daily. Preferred is that the cancer is gastric cancer and that the compound or the salt thereof is administered at a dose of 10 mg once daily. Preferred is that the cancer is gastric cancer and that the compound or the salt thereof is administered at a dose of 10 mg twice daily. Preferred is that the cancer is gastric cancer and that the compound or the salt thereof is administered at a dose of 20 mg once daily. Preferred is that the cancer is gastric cancer and that the compound or the salt thereof is administered at a dose of 20 mg twice daily. Preferred is that the cancer is gastric cancer and that the compound or the salt thereof is administered at a dose of 40 mg once daily. Preferred is that the cancer is gastric cancer and that the compound or the salt thereof is administered at a dose of 25 mg once daily. Preferred is that the cancer is gastric cancer and that the compound or the salt thereof is administered at a dose of 25 mg twice daily. Preferred is that the cancer is gastric cancer and that the compound or the salt thereof is administered at a dose of 35 mg once daily. Preferred is that the cancer is gastric cancer and that the compound or the salt thereof is administered at a dose of 35 mg twice daily. Preferred is that the cancer is gastric cancer and that the compound or the salt thereof is administered at a dose of 70 mg once daily. Preferred is that the cancer is gastric cancer and that the compound or the salt thereof is administered at a dose of 50 mg once daily. Preferred is that the cancer is gastric cancer and that the compound or the salt thereof is administered at a dose of 50 mg twice daily. Preferred is that the cancer is gastric cancer and that the compound or the salt thereof is administered at a dose of 100 mg once daily.

As used herein, “treat”, “treating” or “treatment” refers to restraining, slowing, stopping, or reversing the progression or severity of an existing symptom or disorder.

As used herein, the term “patient” refers to a human which is afflicted with a particular disease, disorder, or condition. As used herein, the term “2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol” refers to a compound whose structure is:

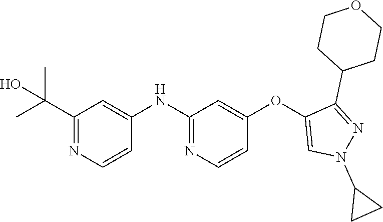 [SMILES: CC(C)(O)c1cc(Nc2cc(Oc3cn(C4CC4)nc3C3CCOCC3)ccn2)ccn1]

It will be understood by the skilled reader that 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol is capable of forming salts. The compounds of the present invention are basic heterocycles, and accordingly react with any of a number of inorganic and organic acids to form pharmaceutically acceptable acid addition salts. Such pharmaceutically acceptable acid addition salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2008); S. M. Berge, et al., “Pharmaceutical Salts,” Journal of Pharmaceutical Sciences, Vol 66, No. 1, January 1977.

The compounds of the present invention are preferably formulated as pharmaceutical compositions administered by a variety of routes. Most preferably, such compositions are for oral or intravenous administration. Such pharmaceutical compositions and processes for preparing same are well known in the art. See, e.g., REMINGTON: THE SCIENCE AND PRACTICE OF PHARMACY (D. Troy, et al., eds., 22nd ed., Lippincott Williams & Wilkins, 2012).

As used herein, the term “effective amount” refers to the amount or dose of compound of the invention, or a pharmaceutically acceptable salt thereof which, upon single or multiple dose administration to the patient, provides the desired effect in the patient under diagnosis or treatment.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

### Preclinical Toxicology Studies

Due to the cardiac effects observed with daily dosing in preclinical models, intermittent-dosing toxicology studies administering 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate for 3 months in dogs and rats were completed to support clinical dosing. The two dosing schedules evaluated were 2 weeks of daily dosing followed by 2 weeks of no dosing (2 weeks on/2 weeks off) or 3 weeks of daily dosing followed by 1 week of no-dosing (3 weeks on/1 week off). There were no adverse effects in dogs administered 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate intermittently for 3 months, and the no-observed-adverse-effect level (NOAEL) was 7.5 mg/kg. Cardiac effects in the rat were limited to aortic inflammation and degeneration in a single female animal administered 12 mg/kg for 3 cycles of the 3-week-on/1-week-off schedule, with no compound-related, adverse effects detected in rats administered 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate for 3-months using the 2-week on/2-week-off dosing schedule. Margins of safety based on the NOAELs for the 3-month studies support the proposed clinical dose range of 10 to 100 mg in patients.

### A Phase 1 Study of 2-{4-[(4-{[1-Cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate in Patients with Solid Tumors

This Phase 1 multicenter, nonrandomized, open-label, first-in-human study will consist of multiple parts including dose escalation (Part A) and monotherapy expansion in glioma (Part B). Part A will consist of a dose-escalation assessment of the safety, tolerability, and PK of 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate in patients who have advanced or metastatic cancer. This part will consist of 2 different dosing schedules (2 weeks on/2 weeks off and 3 weeks on/1 week off). Part B will be an expansion to evaluate 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate at the Part A-selected dose for each schedule in patients who have isocitrate dehydrogenase [IDH]-mutant glioma.

In Part A, a cycle length for both schedules is 28 days. Each cohort will contain a minimum of 3 patients who are evaluable for dose-limiting toxicity (DLT) assessment. Dose escalation to Cohort 2 will be guided by safety assessments in Cohort 1 for each schedule and then further escalations from Cohort 2 will be guided by both PK and safety assessments. Accumulated data from all previous cohorts will be used to evaluate the current dose-escalation scheme and dosing schedules. The highest allowable doses to be studied will not exceed 100 mg/day on the 2 weeks on treatment/2 weeks off treatment and 50 mg/day on the 3 weeks on treatment/1 week off treatment, respectively, unless the AUCs are less than predicted at these doses based on the nonclinical PK model. The dose limiting toxicity (DLT) observation period will last to the completion of Cycle 1, unless a patient discontinues from study treatment during Cycle 1 due to a DLT. The maximum safe dose for each schedule is the maximum of the maximum tolerated dose (MTD) and highest allowable dose and will be the recommended doses for each schedule.

1. A method of treating cancer in a patient comprising administering to the patient an effective amount of a compound that is 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol or a pharmaceutically acceptable salt thereof daily for at least 14 days of a 28-day cycle.

2. The method according to claim 1 wherein the administration is daily for 14 days of 28-day cycle.

3. The method according to claim 1 wherein the administration is daily for 21 days of a 28-day cycle.

4. The method according to claim 1 wherein the compound or the salt thereof is 2-{4-[(4-{[1-cyclopropyl-3-(tetrahydro-2H-pyran-4-yl)-1H-pyrazol-4-yl]oxy}pyridin-2-yl)amino]pyridin-2-yl}propan-2-ol 4-methylbenzenesulfonate.

5. The method according to claim 1 wherein the cancer is colorectal cancer, melanoma, hepatocellular carcinoma, renal cancer, brain cancer, pancreatic cancer, myelodysplastic syndrome, lung cancer, breast cancer, head and neck cancer, or gastric cancer.

6. (canceled)

7. The method according to claim 5 wherein the colorectal cancer is colon cancer.

8. The method according to claim 5 wherein the colorectal cancer is rectal cancer.

9.-12. (canceled)

13. The method according to claim 5 wherein the cancer is brain cancer with an IDH1 mutation.

14. The method according to claim 5 wherein the cancer is brain cancer with an IDH2 mutation.

15.-20. (canceled)

21. The method according to claim 1 wherein the compound or the salt thereof is administered at a dose of 5 mg once daily.

22. The method according to claim 1 wherein the compound or the salt thereof is administered at a dose of 5 mg twice daily.

23. The method according to claim 1 wherein the compound or the salt thereof is administered at a dose of 10 mg once daily.

24. The method according to claim 1 wherein the compound or the salt thereof is administered at a dose of 10 mg twice daily.

25. The method according to claim 1 wherein the compound or the salt thereof is administered at a dose of 20 mg once daily.

26. The method according to claim 1 wherein the compound or the salt thereof is administered at a dose of 20 mg twice daily.

27. The method according to claim 1 wherein the compound or the salt thereof is administered at a dose of 40 mg once daily.

28. The method according to claim 1 wherein the compound or the salt thereof is administered at a dose of 25 mg once daily.

29. The method according to claim 1 wherein the compound or the salt thereof is administered at a dose of 25 mg twice daily.

30. The method according to claim 1 wherein the compound or the salt thereof is administered at a dose of 35 mg once daily.

31. The method according to claim 1 wherein the compound or the salt thereof is administered at a dose of 35 mg twice daily.

32. The method according to claim 1 wherein the compound or the salt thereof is administered at a dose of 70 mg once daily.

33. The method according to claim 1 wherein the compound or the salt thereof is administered at a dose of 50 mg once daily.

34. The method according to claim 1 wherein the compound or the salt thereof is administered at a dose of 50 twice daily.

35. The method according to claim 1 wherein the compound or the salt thereof is administered at a dose of 100 mg once daily.

36.-70. (canceled)
